(19) United States
(12) Patent Application Publication
(10) Pub. No.: US 2015/0272772 A1
(43) Pub. Date: Oct. 1, 2015

(54) Medical Securing Unit and Medical Imaging Apparatus with the Medical Securing Unit

(71) Applicants: Wolfgang Kraus, Furth (DE); Jürgen Peter, Ebermannstadt (DE); Stephan Zink, Erlangen (DE)

(72) Inventors: Wolfgang Kraus, Furth (DE); Jürgen Peter, Ebermannstadt (DE); Stephan Zink, Erlangen (DE)

(21) Appl. No.: 14/671,080

(22) Filed: Mar. 27, 2015

(30) Foreign Application Priority Data
Mar. 28, 2014 (DE) 102014205837.6

(51) Int. Cl. A61F 5/37; A61B 5/055; A61B 6/03; A61B 6/04

(52) U.S. Cl. CPC A61F 5/3776; A61B 6/0421; A61B 5/0555; A61B 6/032; A61B 6/037

(57) ABSTRACT

A medical securing unit is configured to secure the position of a patient and/or a medical accessory unit. The medical securing unit has at least one securing belt element and a belt stowing unit.

This application claims the benefit of DE 102014205837.6, filed on Mar. 28, 2014, which is hereby incorporated by reference in its entirety.

### BACKGROUND

In a medical imaging examination performed on a patient using a medical imaging apparatus, the patient is positioned on a patient couch and moved together with the patient couch into a patient examination region of the medical imaging apparatus. The patient is secured on the patient couch by securing belts for his/her protection. Medical accessory units for the medical imaging examination are secured together with the patient using the securing belts. These medical accessory units may include for example local coil units for a medical magnetic resonance examination and/or an EKG unit, etc.

Such securing belts are configured such that they may be used for patients having different volumes. However this may mean that securing belt regions not required to secure the patient and/or protruding securing belt regions may get in the way when the patient couch is being moved.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, the disclosed embodiments may provide a medical securing unit, with which securing belt regions that are not required may be stowed safely.

The disclosed embodiments include a medical securing unit configured to secure the position of a patient and/or a medical accessory unit and that has at least one securing belt element.

In one aspect, the medical securing unit has a belt stowing unit. In this embodiment, a subregion of the securing belt element not required to secure the patient and/or to secure medical accessory units may be stored safely within the belt stowing unit, thereby preventing protrusion of the at least one securing belt element. Safety may be improved when a patient support apparatus, e.g., the table together with the patient, is being moved, as obstruction by securing belt elements for securing the patient on the table, which are hanging down, may be prevented. It also allows the patient to be safely introduced into a patient examination region of a medical imaging apparatus, for example a magnetic resonance apparatus, a PET apparatus, a computed tomography apparatus, etc., together with the table of the patient support, as obstruction of an inward movement of the table by securing belt elements that are hanging down may be prevented.

Safe stowing of the securing belt element may be achieved if the at least one securing belt element has two opposing end regions in the longitudinal extension of the securing belt element and one of the end regions of the at least one securing belt element is arranged within the belt stowing unit. One end region of the securing belt element may be fixed within the belt stowing unit so that the securing belt element is prevented from falling out of the belt stowing unit.

The belt stowing unit may include a belt retraction unit. This allows the subregion of the securing belt element not required to secure the patient and/or a medical accessory unit to be stored in a space-saving manner within the belt stowing unit. Also a subregion of the securing belt element projecting out of the belt stowing unit may be adjusted to a length of the securing belt element involved in fastening and/or securing the patient and/or the accessory unit. The securing belt element may be removed easily from the belt stowing unit, thereby simplifying a workflow and/or work sequence, for example to prepare a patient for a medical imaging examination, optimizing time.

In one embodiment, the belt stowing unit has a housing unit and the belt retraction unit is supported rotatably on the housing unit within a space enclosed by the housing unit. This allows the securing belt element to be arranged with a good level of protection within the belt stowing unit. For example it allows the securing belt element and also the belt retraction unit to be protected from dirt and/or damage.

A subregion of the securing belt element not required may be rolled up easily within the belt stowing unit, if the belt retraction unit has at least one retraction element for generating a torque.

The retraction element may include a spring element, such as a spring element made of a rubber material, for example a rubber strip. This allows a low-cost belt stowing unit to be provided. The spring element may be configured as non-magnetic, so the medical securing unit may be used with a magnetic resonance apparatus.

In one aspect, the belt retraction unit has at least one cylindrical rolling body. This allows easy rolling up and/or unrolling of the securing belt element to be achieved. It also allows fast access to the securing belt element arranged within the belt stowing unit.

The cylindrical rolling body may have an internal hollow space and the at least one retraction element is arranged within the hollow space. This allows the retraction element to be stored within the belt stowing unit with a good level of protection from the securing belt element. It also prevents obstruction of the movement of the retraction element by the securing belt element, thereby reducing wear of the belt stowing unit.

The at least one retraction element may have two opposing end regions along its longitudinal extension, with a first end region of the at least one retraction element being arranged on the cylindrical rolling body and a second end region being arranged on a housing unit of the belt stowing unit. This allows a torque generated by the retraction element to be transmitted easily to the cylindrical rolling body, so that it may execute a rotational movement about a rotation axis, which may be parallel to its longitudinal extension, relative to the housing unit of the belt stowing unit. It also allows bracing of the retraction element against the housing unit to generate and/or transmit the torque.

In one embodiment, the belt retraction unit has at least two retraction elements, which are arranged one behind the other within the cylindrical rolling body. This allows translatory movement of the cylindrical rolling body, allowing a location and/or position, e.g., a latching position of the cylindrical rolling body, in which a rotational movement of the cylindrical rolling body is prevented in at least one rotation direction, to be set.

The first retraction element of the at least two retraction elements may be configured differently from the second retraction element of the two retraction elements in respect of a physical property and/or a material property. The cylindrical rolling body may be supported at least partially contactlessly in relation to the housing unit, as the cylindrical rolling body is braced against the housing unit at least partially by the at least two retraction elements. The first retraction element and the second retraction element may be of identical length but may differ in respect of a spring constant and/or a modulus of elasticity. The first retraction element and the second retraction element may also have an identical spring constant and/or an identical modulus of elasticity but may differ in length.

In one embodiment, the belt stowing unit has at least one locking element, by which a retraction position of the belt retraction unit may be locked. This may prevent a rotational movement of the belt retraction unit, e.g., of the rolling body of the belt retraction unit, along at least one rotation direction, thus preventing the unrolling and/or rolling up of the securing belt element, e.g., in a securing position for securing the patient and/or medical accessory units. The locking element here may be formed by a latching element and/or a clamping element and/or by further locking elements that appear expedient to the person skilled in the art.

The belt stowing unit may have an actuating element, with a locking position of the at least one locking element being set as a function of an actuation position of the actuating element relative to a housing unit of the belt stowing unit. This allows a user-friendly embodiment of the medical securing unit to be achieved. The actuating element may include for example a tiltable lever, which effects a displacement of the rolling body along its longitudinal extension and thus effects locking, e.g., clamping, between the rolling body and the housing unit. Alternatively or additionally, the actuating element may include a push button, which for example only allows a rotational movement of the rolling body when pushed in and results in locking, e.g., a latching connection, between the rolling body and the housing unit when not pushed in. The actuating element may also include a push button together with a cardioid slide, which similarly allows a rotational movement of the rolling body in one position and effects locking between the rolling body and the housing unit in a further position.

The medical securing unit may have at least one fastening element, with the result that the medical securing unit may be arranged on further units to secure the patient and/or medical accessory units. The medical securing unit may also be arranged directly on the patient support apparatus and/or on a medical accessory unit. This allows the medical securing unit to be arranged detachably on the further units.

One distinction from different medical securing units may be achieved, if the medical securing unit has a housing unit with at least one marking element, the at least one marking element indicating an available belt length.

In one aspect, a medical accessory unit includes at least one medical securing unit. The at least one medical securing unit is configured to secure a position of a patient and/or the medical accessory unit and to this end has at least one securing belt element and a belt stowing unit. The medical accessory unit allows this to be positioned on a patient and/or on a patient support apparatus safely for a medical imaging examination. The medical accessory unit may include a magnetic resonance coil unit, an EKG unit, an infusion unit and/or further medical accessory units that may be expedient and are involved in a medical imaging examination. The features of the inventive medical accessory unit may correspond to the features of the medical securing unit, which are set out in detail above. Features, advantages or alternative embodiments mentioned here may also be applied to other claimed subject matter and vice versa.

The medical accessory unit may have a fastening element configured to correspond to a fastening element of the at least one medical securing unit. This allows the medical securing unit to be arranged quickly on the medical accessory unit to secure the medical accessory unit, for example by a latching connection between the two fastening elements. Alternatively the medical accessory unit may also be configured such that the at least one medical securing unit is already contained in a housing unit of the medical accessory unit, so that a space-saving arrangement of the at least one medical securing unit may be achieved.

In one aspect, a patient support apparatus includes at least one medical securing unit. The medical securing unit is configured to secure a position of a patient and/or a medical accessory unit and to this end has at least one securing belt element and a belt stowing unit. The patient support apparatus allows a patient positioned on the patient support apparatus and/or a medical accessory unit positioned on the patient support apparatus to be positioned safely for, e.g., a medical imaging examination. The features of the patient support apparatus may correspond to the features of the medical securing unit, which are set out in detail above. Features, advantages or alternative embodiments mentioned here may also be applied to other claimed subject matter and vice versa.

The patient support apparatus may have a table for supporting a patient, the table having at least one fastening element to fasten the at least one medical securing unit to the table. This allows the medical securing unit to be arranged on the patient support apparatus quickly to secure the patient and/or a medical accessory unit. The fastening element of the table may be configured to correspond to a fastening element of the medical securing unit.

In one embodiment, the fastening element has a fastening rail. This allows variable positioning of the medical securing unit. A position of the medical securing unit on the table may be tailored to a position of an object to be secured, for example the patient and/or the medical accessory unit. The table may have two fastening rails, which may be arranged on a lateral edge region of the table. The medical securing unit may be supported in a movable manner by a fastening element corresponding to the fastening rail within the fastening rail of the table. The medical securing unit may be supported in a movable manner completely within the fastening rail and may be able to be fixed in position by a fastening element of the medical securing unit.

Alternatively the table of the patient support apparatus may be configured such that the table includes the at least one medical securing unit, in other words the at least one medical securing unit is integrated directly in the table. A housing of the at least one medical securing unit and a housing of the table for example may be configured as a single piece.

In one aspect, a medical imaging apparatus includes a patient support apparatus, which includes a table to support a patient, and at least one medical securing unit with at least one securing belt element to secure the patient and/or a medical accessory unit on the table. The medical securing unit has a belt stowing unit. The features of the medical imaging apparatus may correspond to the features of the medical securing unit, which are set out in detail above. Features, advantages or alternative embodiments mentioned may also be applied to other claimed subject matter and vice versa.

The at least one medical securing unit may be configured as separate from the patient support apparatus and/or separate from the medical accessory unit. The at least one inventive medical securing unit may be contained in the patient support apparatus and/or the medical accessory unit.

### BRIEF DESCRIPTION OF THE DRAWINGS

FIG. 1 shows a schematic diagram of a medical securing unit in accordance with one embodiment.

FIG. 2 shows a section through the medical securing unit illustrated in FIG. 1.

FIG. 3 shows a schematic diagram of a medical securing unit in accordance with one embodiment.

FIG. 4 shows a schematic diagram of a medical securing unit in accordance with one embodiment.

FIG. 5 shows a plan view of a patient support apparatus with a medical securing unit in accordance with one embodiment.

FIG. 6 shows a sectional diagram of a patient support apparatus with a medical securing unit in accordance with one embodiment.

FIG. 7 shows a sectional diagram of a patient support apparatus with a medical securing unit in accordance with one embodiment.

FIG. 8 shows a plan view of a medical accessory unit with a medical securing unit in accordance with one embodiment.

FIG. 9 shows a sectional diagram of a medical accessory unit with a medical securing unit in accordance with one embodiment.

FIG. 10 shows a sectional diagram of a medical accessory unit with a medical securing unit in accordance with one embodiment.

FIG. 11 shows a schematic diagram of a medical imaging apparatus with a medical securing unit in accordance with one embodiment.

### DETAILED DESCRIPTION

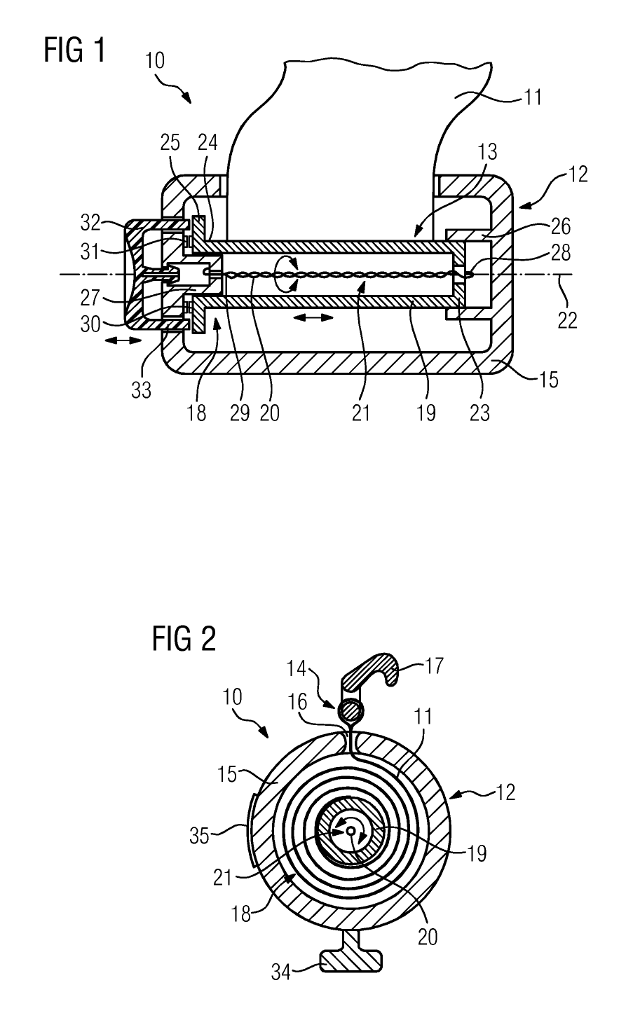

FIGS. 1 and 2 show a medical securing unit 10. The medical securing unit 10 includes a securing belt element 11 for securing a patient 701 and/or a medical accessory unit 500, 600 on a patient support apparatus 300, 400 for a medical imaging examination, such as for example a magnetic resonance examination, a computed tomography examination, a PET examination (positron emission tomography examination) etc. For safe stowing of a subregion of the securing belt element 11 not required to secure the patient 701 and/or the medical accessory unit 500, 600, the medical securing unit 10 has a belt stowing unit 12.

The securing belt element 11 has two opposing end regions 13, 14 in the longitudinal extension of the securing belt element, with a first end region 13 being arranged within the belt stowing unit 12. To this end the end region 13 of the securing belt element 11 arranged within the belt stowing unit 12 is fixed within the belt stowing unit 12. The belt stowing unit 12 also has a housing unit 15, which has a web-type recess 16. A second end region 14 of the two opposing end regions 13, 14 of the securing belt element 11 in this example projects out of the web-type recess 16 of the housing unit 15. Also arranged on the second end region 14 of the securing belt element 11 is a fastening element 17. In the present exemplary embodiment the fastening element 17 includes a hook (FIG. 2). The fastening element 17 may also be formed by a Velcro fastener, a latching element and/or further fastening elements 17.

For space-saving stowing of the securing belt element 11 within the belt stowing unit 12, the belt stowing unit 12 has a belt retraction unit 18, which is arranged within a region of the belt retraction unit 12 enclosed by the housing unit 15. The belt retraction unit 18 in this example is supported rotatably within the belt stowing unit 12 on the housing unit 15. To this end the belt retraction unit 18 has a cylindrical rolling body 19 and a retraction element 20. The retraction element 20 in this example generates a torque, which is transmitted to the cylindrical rolling body 19, for the turning and/or rotation of the cylindrical rolling body 19.

The cylindrical rolling body 19 has an internal hollow space 21, in which the retraction element 20 is arranged (FIGS. 1 and 2). The securing belt element 11 is arranged on a cylindrical outer face of the cylindrical rolling body 19. The end region 14 of the securing belt element 11 arranged within the belt stowing unit 12 is fixed with the outer face of the cylindrical rolling body 19 in this example. When the cylindrical rolling body 19 rotates about a rotation axis 22, which is identical to a center axis of the cylindrical rolling body 19, the securing belt element 11 is thus rolled up onto the cylindrical rolling body 19 or unrolled from the cylindrical rolling body 19 (FIG. 2).

The cylindrical rolling body 19 has two opposing end regions 23, 24 along its longitudinal extension. A first end region 23 of the two end regions 23, 24 of the cylindrical rolling body 19 is configured as closed and a second end region 24 of the two end regions 23, 24 of the cylindrical rolling body 19 is configured as open. The cylindrical rolling body 19 also has an edge element 25 on the second, open end region 24, the edge element 25 extending outward in a radial direction in the manner of a web. The cylindrical rolling body 19 is also arranged within the belt stowing unit 12 such that the longitudinal extension of the cylindrical rolling body 19 is parallel to a longitudinal extension of the web-type recess 16 of the housing unit 15.

The housing unit 15 has a ring element 26 on a side facing the closed end region 23 of the cylindrical rolling body 19, the ring element 26 extending in the manner of a web from the side facing the cylindrical rolling body 19 in the direction of the cylindrical rolling body 19. The ring element 26 has an internal diameter, which is greater than an external diameter of the cylindrical rolling body 19. The internal diameter of the ring element 26 in this example corresponds essentially to the external diameter of the cylindrical rolling body 19 and a tolerance range to compensate for movement, so that the closed end region 23 of the cylindrical rolling body 19 is supported in a movable manner within the ring element 26. The ring element 26 only extends so far into the interior of the housing unit 15 that the rolling up of the securing belt element 11 by the cylindrical rolling body 19 is not impeded by the ring element 26.

On a side of the housing unit 15 facing the open end region 24 of the cylindrical rolling body 19 the housing unit 15 has a cylindrical web region 27, which extends from the side facing the open end region 24 of the cylindrical rolling body 19 in the direction of the cylindrical rolling body 19. The cylindrical web region 27 of the housing unit 15 has an external diameter, which is smaller than an internal diameter of the cylindrical rolling body 19. The internal diameter of the cylindrical rolling body 19 in this example corresponds essentially to the external diameter of the cylindrical web region 27 and a tolerance range to compensate for movement, so that the open end region 24 of the cylindrical rolling body 19 is supported in a movable manner on the cylindrical web region 27 of the housing unit 15.

The cylindrical rolling body 19 has a length, which is shorter than the length of a space enclosed by the housing unit 15. This allows the cylindrical rolling body 19 to execute a translatory movement along its longitudinal extension, as is useful when locking the cylindrical rolling body 19 and releasing the cylindrical rolling body 19 from the locking position.

In the present exemplary embodiment, the belt stowing unit has a single retraction element 20 (FIGS. 1 and 2). The retraction element 29 includes a spring element, which is formed from a rubber material, so that the retraction element 20 is configured as non-magnetic so the medical securing unit 10 may be used during a magnetic resonance examination. The spring element in this example may have the form of a rubber strip and/or other forms. The spring element, in the present exemplary embodiment the rubber strip, is arranged within the internal hollow space 21 of the cylindrical rolling body 19. A rotational movement of the cylindrical rolling body 19 causes the rubber strip to be arranged in an at least partially twisted manner within the hollow space 21.

The twisted rubber strip has two end regions 28, 29 along its longitudinal extension, a first end region 28 of the rubber strip being arranged on the closed end region 23 of the cylindrical rolling body 19. The second end region 29 of the rubber strip is arranged on the housing unit 15 of the belt stowing unit 12. The rubber strip is arranged on the cylindrical web region 27 in this example. The rubber strip is also arranged centrally within the cylindrical rolling body 19, so that the rubber strip is enclosed coaxially by the cylindrical rolling body 19 (FIGS. 1 and 2).

The belt stowing unit 12 also has two locking elements 30, 31, by which a retraction position of the belt retraction unit 18, e.g., of the cylindrical rolling body 19 in relation to the housing unit 15, may be locked. A first locking element 30 is arranged on the cylindrical rolling body 19. The first locking element 30 in this example is arranged on the open end region 24 of the cylindrical rolling body 19, with the first locking element 30 being arranged on a side of the edge element 25 of the cylindrical rolling body 19 facing the housing unit 15. The second locking element 31 is arranged on the side of the housing unit 15 facing the open end region 23 of the cylindrical rolling body 19.

The two locking elements 30, 31 are configured as annular and have an identical diameter, so that, when the cylindrical rolling body 19 is in contact with the housing unit 15, the two locking elements 30, 31 enter into a locking connection to lock the cylindrical rolling body 19 relative to the housing unit 15. To this end, the two locking elements 30, 31 are configured to correspond to one another. For example, the two locking elements 30, 31 may be formed by corresponding latching elements or corresponding clamping elements. Other embodiments of the two locking elements 30, 31 may be used.

The belt stowing unit 12 also has an actuating element 32, formed in the present exemplary embodiment by a push button. The actuating element 32 is arranged on a subregion of the housing unit 15 facing the open end region 24 of the cylindrical rolling body 19. The actuating element 32 is configured as cylindrical and an open end region faces the housing unit 15. Segments of an edge region 33 of the actuating element 32 project through the housing unit 15, so that when the actuating element 32 is actuated the actuating element 32 executes a movement in a direction that is essentially parallel to the longitudinal extension of the cylindrical rolling body 19. To this end, the housing unit 15 has recesses, through which the edge region 33 of the actuating element 32 may project. The edge region 33 in this example rests against a side of the edge element 25 of the open end region 24 of the cylindrical rolling body 19 facing the housing unit 15. The actuating element 32 also has a securing element, which extends away from the actuating element 32 and is arranged in the cylindrical web region 27 of the housing unit 15, so that the actuating element 32 is supported safely on the housing unit 15.

When the actuating element 32 is actuated, for example when an operator pushes the actuating element 32, a force is transmitted from the actuating element 32 to the edge element 25 of the cylindrical rolling body 19. This force effects a movement of the cylindrical rolling body 19 in a direction away from the actuating element 32, such movement of the cylindrical rolling body 19 taking place counter to a spring force of the rubber strip. This causes the two locking elements 30, 31 to release their lock and the cylindrical rolling body 19 may be rotated about its center axis. If the entire securing belt element 11 is rolled up on the cylindrical rolling body 19, in other words only the second end region 14 of the securing belt element 11 projects from the belt stowing unit 12, only one rotation direction of the cylindrical rolling body 19 is possible, allowing the securing belt element 11 to be unrolled. If the securing belt element 11 is at least partially unrolled, for example if an operator pulls on the end region 14 of the securing belt element 11 projecting from the belt stowing unit 12, this effects a rotation of the cylindrical rolling body 19, thereby twisting the rubber strip. The twisting of the rubber strip causes rotation energy to be stored in the rubber strip, generating a torque for a counter movement of the cylindrical rolling body 19. If the operator were to release the securing belt element 11, the cylindrical rolling body 19 would execute a rotational movement due to the torque of the rubber strip, bringing about the rolling up of the securing belt element 11.

If the actuating element 32 is in a not-pushed actuation position, the spring force of the rubber strip also causes the cylindrical rolling body 19 to move in the direction of the cylindrical web region 27, so that the two locking elements 30, 31 of the cylindrical rolling body 19 and of the housing unit 15 enter into a locking position with one another. In this position, rotation of the cylindrical rolling body 19 relative to the housing unit 15 is blocked. A force for moving the cylindrical rolling body 19 in a direction of the cylindrical web region 27 is produced in this case by a spring force of the rubber strip, which is deflected counter to the spring force when the actuating element 32 is pushed in.

In the present exemplary embodiment, the medical securing unit 10 has a fastening element 34. The medical securing unit 10 may be fastened to a medical accessory unit 500, 600 and/or to a patient support apparatus 300, 400 by the fastening element 34 (FIG. 2). In this case, the fastening element 34 is configured as T-shaped. Other embodiments of the fastening element 34 may be used.

The medical securing unit 10 also has a marking element 35, which is arranged on an outer face of the housing unit 15 (FIG. 2). The marking element 35 indicates the available belt length of the securing belt element 11. The marking element 35 may include for example an “S”, “M”, “L”, “XL”, etc. to identify the available belt length of the securing belt element 11.

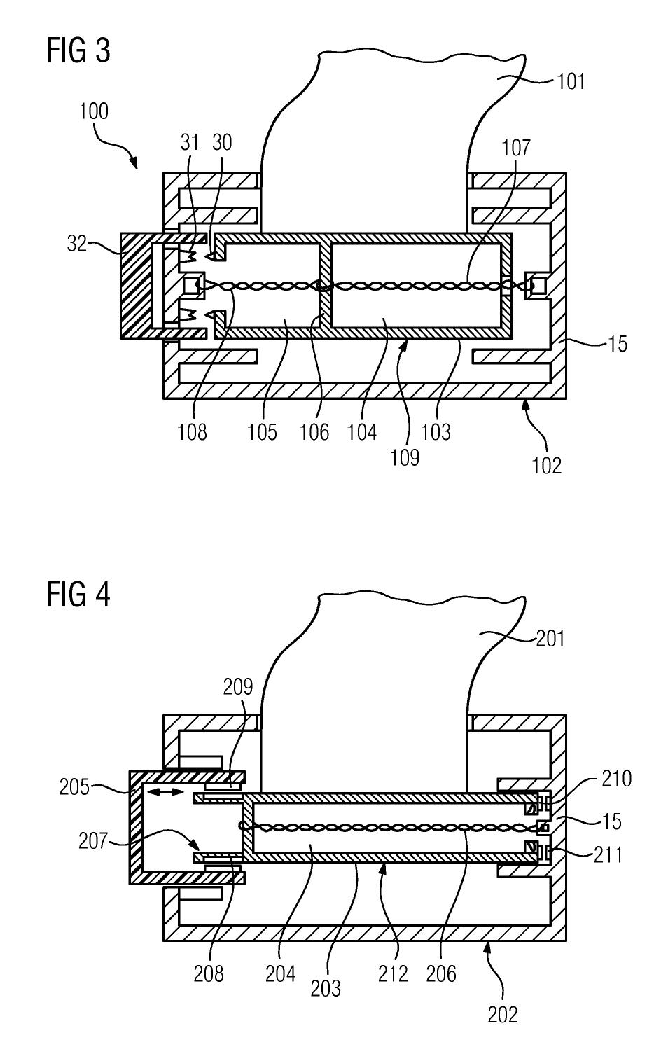

FIG. 3 shows an exemplary embodiment of the medical securing unit 100. Essentially identical components, features and functions are shown with identical reference characters. The description that follows is limited essentially to the differences from the exemplary embodiment in FIGS. 1 and 2, with reference being made to the description of the exemplary embodiment in FIGS. 1 and 2 for identical components, features and functions.

The medical securing unit 100 has a securing belt element 101 and a belt stowing unit 102, with the securing belt element 101 being arranged at least partially within the belt stowing unit 102. The belt stowing unit 101 has a belt retraction unit 109 configured differently from FIGS. 1 and 2 with a cylindrical rolling body 103. The cylindrical rolling body 103 has two hollow spaces 104, 105, which are enclosed by a cylindrical outer surface of the cylindrical rolling body 103, with the two hollow spaces 104, 105 being arranged one behind the other along a longitudinal extension of the cylindrical rolling body 103. The two hollow spaces 104, 105 are separated from one another by a separating wall 106 of the cylindrical rolling body 103.

The belt retraction unit 109 also has two retraction elements 107, 108, with one of the two retraction elements 107, 108 arranged in each of the two hollow spaces 104, 105 of the cylindrical rolling body 103, so that the two retraction elements 107, 108 are arranged one behind the other within the cylindrical rolling body 103. The two retraction elements 107, 108 are configured as described in connection with FIGS. 1 and 2 and are formed by rubber strips. Each of the two rubber strips is arranged on the separating wall 106 of the cylindrical rolling body 103 and also on the housing unit 15. In the present exemplary embodiment, the two hollow spaces 104, 105 and therefore also the two retraction elements 107, 108 are of different length. This allows a different spring force to be induced in the two retraction elements 104, 105 by the two different retraction elements 107, 108.

The first hollow space 105 and the first retraction element 108, which face the actuating element 32, are shorter in length than the second hollow space 104 and the second retraction element 107, which are arranged on a side of the cylindrical rolling body 103 facing away from the actuating element 32. Rotation of the cylindrical rolling body 103 causes the two retraction elements 107, 108 configured as rubber strips to be twisted. Because the first retraction element 108 is shorter than the second retraction element 107, a greater spring force is generated in the first retraction element 108 than in the second retraction element 107. When the actuating element 32 is released, this causes the cylindrical rolling body 103 to move in the direction of the actuating element 32 and be made to lock with the housing unit 15 by the locking elements 30, 31.

Alternatively, the first and second hollow space 104, 105 and therefore also the first and second retraction elements 107, 108 may be of identical length. However, in order to achieve different spring forces in the two retraction elements 107, 108, the first and second retraction elements 107, 108 differ in respect of their spring constant and/or their modulus of elasticity.

The further embodiment of the medical securing unit 200 corresponds to the descriptions relating to FIGS. 1 and 2. FIG. 4 shows an exemplary embodiment of the medical securing unit 200. Essentially identical components, features and functions are shown with identical reference characters. The description that follows is limited essentially to the differences from the exemplary embodiment in FIGS. 1 and 2, with reference being made to the description of the exemplary embodiment in FIGS. 1 and 2 for identical components, features and functions.

The medical securing unit 200 has a securing belt element 201 and a belt stowing unit 202, with the securing belt element 201 being arranged at least partially within the belt stowing unit 202. The belt stowing unit 202 has a belt retraction unit 212 configured differently from FIGS. 1 and 2 with a cylindrical rolling body 203. The cylindrical rolling body 203 has a hollow space 204. The belt stowing unit 102 also has an actuating element 205, with the hollow space 204 of the cylindrical rolling body 203 being configured as open on a side facing away from the actuating element 205.

A retraction element 206 of the belt retraction unit 212 is configured as a rubber strip and arranged on the cylindrical rolling body 203 on an end facing the actuating element 204 and arranged on the housing unit 15 on an end facing away from the actuating element 205. The cylindrical rolling body 203 has an end region 207 facing the actuating element 205, with a cardioid slide 208 (not shown in detail) arranged on this end region 207. A runner element (not shown in detail) corresponding to a latching element 209 of the actuating element 205 engages in the cardioid slide 208. When the actuating element 205 is actuated, e.g., when the actuating element 205 is pulled out, by an operator, a latching connection is established between the actuating element 205 and the cylindrical rolling body 203, so that the cylindrical rolling body 203 may be rotated about its longitudinal axis by an operator by the actuating element 205.

The cylindrical rolling body 203 has an end region facing away from the actuating element 205, on which a locking element 210 is arranged on a side facing the housing unit 15. A locking element 211 corresponding to the locking element 210 of the cylindrical rolling body 203 is also arranged on the housing unit 15, so that in a decoupled state there is locking of a set position of the cylindrical rolling body 203 between the cylindrical rolling body 203 and the actuating element 205.

The further embodiment of the medical securing unit 200 corresponds to the descriptions relating to FIGS. 1 and 2.

FIGS. 5 to 7 each show a patient support apparatus 300, 400 with at least one medical securing unit 10, 100, 200. The medical securing units 10, 100, 200 shown in FIGS. 5 to 7 are configured according to one of the embodiments shown in FIGS. 1 to 4.

The patient support apparatus 300 in FIG. 5 has a table 301, on which a patient is positioned for a medical imaging examination. To secure the patient and/or to secure medical accessory units, which are required for the medical imaging examination, for example a magnetic local coil unit, an EKG unit, etc., the patient support apparatus 300 includes a number of medical securing units 10, 100, 200. In the present exemplary embodiment, the patient support apparatus 300 has four medical securing units 10, 100, 200. The number of medical securing units 10, 100, 200 may be tailored to a specific application such that more than four or fewer than four medical securing units 10, 100, 200 may be provided.

To arrange the medical securing units 10, 100, 200 on the patient support apparatus 300, the patient support apparatus 300 has two fastening elements 302, which are configured in the manner of channels. The two fastening elements 302 are each arranged on an edge region 303 of the table 301, so that a support surface for the patient remains unobstructed. The two fastening elements 303 are also aligned parallel to the longitudinal extension of the table 301.

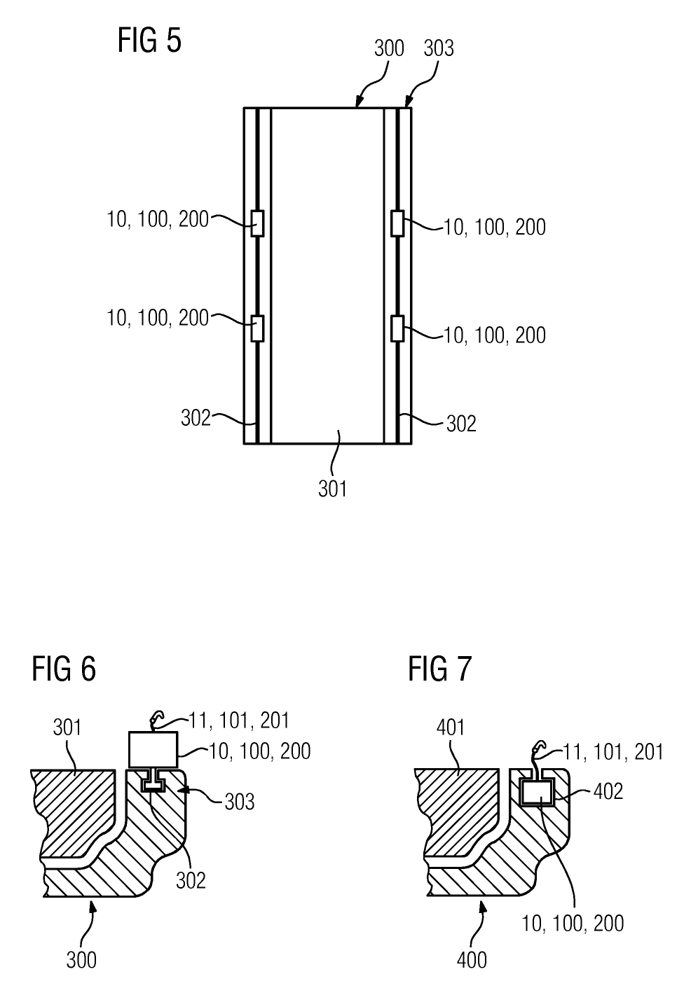

FIG. 6 shows an example of the fastening of one of the medical securing units 10, 100, 200 to the table 301. The two fastening elements 302 each include a T-shaped channel. To this end, the T-shaped fastening element 34 of the medical securing unit 10, 100, 200 is inserted into the T-shaped channel of the table 301. The medical securing unit 10, 100, 200 may be moved along the longitudinal extension of the table 301 within the T-shaped channel, until a desired position for the medical securing unit 10, 100, 200 is reached. A locking element (not shown in detail) may be used to lock the position of the medical securing unit 10, 100, 200 relative to the table 301.

FIG. 7 shows an exemplary embodiment of the patient support apparatus 400. Essentially identical components, features and functions are shown with identical reference characters. The description that follows is limited essentially to the differences from the exemplary embodiment in FIGS. 5 and 6, with reference being made to the description of the exemplary embodiment in FIGS. 5 and 6 for identical components, features and functions.

FIG. 7 shows a section of the patient support apparatus 400 with a table 401 and a fastening element 402 for fastening the medical securing unit 10, 100, 200. The fastening element 302 is configured in the manner of a channel as in FIGS. 5 and 6, the channel being of such a size that the entire medical securing unit 10, 100, 200 may be held in the channel and may be moved along a longitudinal extension of the channel within the channel. The further embodiment of the patient support apparatus 400 corresponds to the descriptions relating to FIGS. 5 and 6.

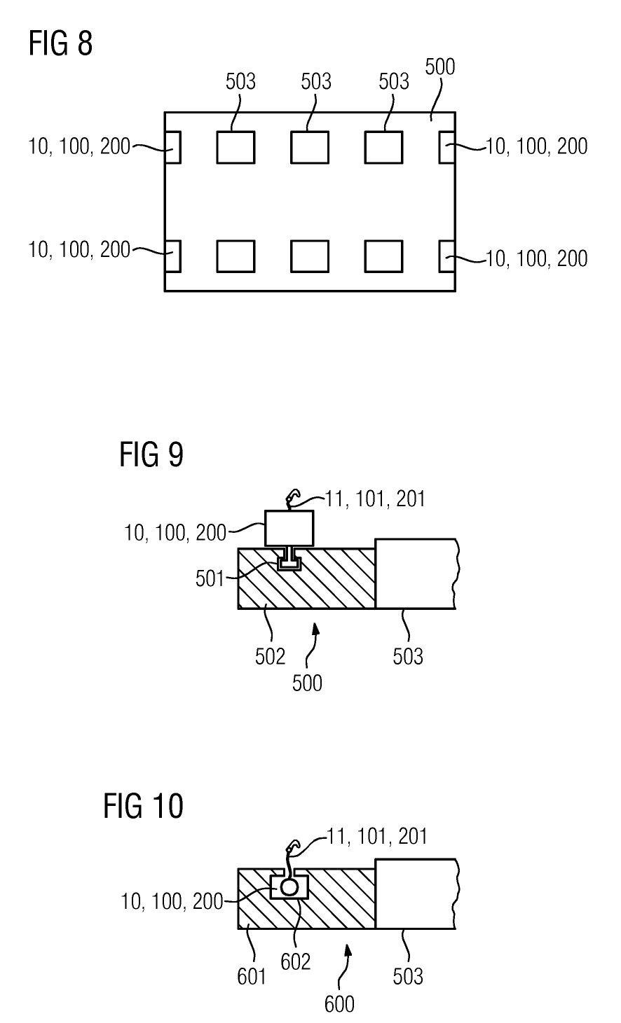

FIGS. 8 to 10 each show a medical accessory unit 500, 600 formed by a magnetic resonance coil apparatus with at least one medical securing unit 10, 100, 200. The medical securing units 10, 100, 200 shown in FIGS. 8 to 10 are configured according to the embodiment shown in FIGS. 1 to 4.

The magnetic resonance coil apparatus in FIG. 8 includes a number of coil elements 503. The magnetic resonance coil apparatus also includes a number of medical securing units 10, 100, 200 for fastening and/or securing the magnetic resonance coil apparatus to a patient. In the present exemplary embodiment, the magnetic resonance coil apparatus has four medical securing units 10, 100, 200. The number of medical securing units 10, 100, 200 may be tailored to a specific application such that more than four or fewer than four medical securing units 10, 100, 200 may be provided.

FIG. 9 shows a section of the magnetic resonance coil apparatus with the medical securing unit 10, 100, 200 in more detail. To this end, the magnetic resonance coil apparatus has a fastening element 501, which is integrated in a housing unit 502 of the magnetic resonance coil apparatus. The fastening element 501 includes an L-shaped recess within the housing unit 502, with the fastening element 34 of the medical securing unit 10, 100, 200 also being configured as L-shaped. To fasten the medical securing unit 10, 100, 200 to the magnetic resonance coil apparatus, the fastening element 34 of the medical securing unit 10, 100, 200 latches into the L-shaped recess of the magnetic resonance coil apparatus.

FIG. 10 shows an exemplary embodiment in the form of a medical accessory unit 600 configured as a magnetic resonance coil apparatus. Essentially identical components, features and functions are shown with identical reference characters. The description that follows is limited essentially to the differences from the exemplary embodiment in FIGS. 8 and 9, with reference being made to the description of the exemplary embodiment in FIGS. 8 and 9 for identical components, features and functions.

FIG. 10 shows a section of the magnetic resonance coil apparatus, in which a housing unit 601 of the magnetic resonance coil apparatus has a recess 602 to hold the medical securing unit 10, 100, 200 in its entirety. The housing unit 601 of the magnetic resonance coil apparatus in this example may be configured as a single piece with the housing unit 15 of the medical securing unit 10, 100, 200.

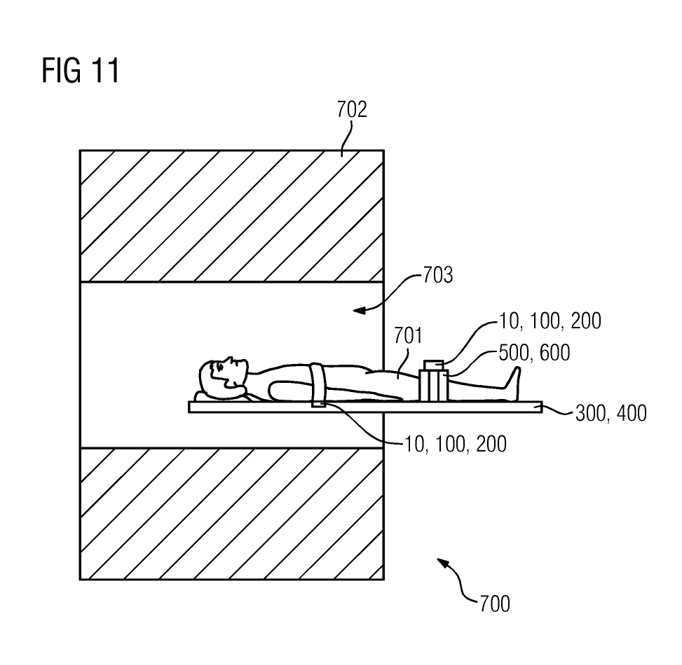

FIG. 11 shows a schematic diagram of a medical imaging apparatus 700, which is formed in the present exemplary embodiment by a magnetic resonance apparatus. Alternatively, the medical imaging apparatus 700 may also include a computed tomography apparatus, a PET apparatus, a C-arm apparatus and/or other configurations of a medical imaging apparatus 700.

The magnetic resonance apparatus includes a magnet unit 702 with a main magnet for generating a powerful and, e.g., constant, main magnetic field. The magnetic resonance apparatus also has a patient receiving region 703 for holding a patient 701. The patient receiving region 703 in the present exemplary embodiment is configured as cylindrical. The patient receiving region 703 is enclosed in a circumferential direction by the magnet unit 702 in the manner of a cylinder. However, a different configuration of the patient receiving region 703 may be provided.

The patient 701 may be moved by a patient support apparatus 300, 400 of the magnetic resonance apparatus into the patient receiving region 703. The illustrated patient support apparatus 300, 400 is configured according to one of the embodiments shown in FIGS. 5 to 7.

For a magnetic resonance examination, a medical accessory unit 500, 600 configured as a magnetic resonance coil apparatus is arranged on the patient. The illustrated magnetic resonance coil apparatus is configured according to the embodiment shown in FIGS. 8 to 10.

To secure the patient 701 and/or medical accessory units 500, 600, the magnetic resonance apparatus has medical securing units 10, 100, 200, the medical securing units 10, 100, 200 illustrated in FIG. 11 being configured according to one of the embodiments shown in FIGS. 1 to 4.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

1. A medical securing unit to secure a position of a patient, a medical accessory unit, or both the patient and the medical accessory unit, the medical securing unit comprising:
at least one securing belt element; and
a belt stowing unit.

2. The medical securing unit of claim 1, wherein the at least one securing belt element has two opposing end regions in the longitudinal extension of the securing belt element and one of the end regions of the at least one securing belt element is disposed within the belt stowing unit.

3. The medical securing unit of claim 1, wherein the belt stowing unit comprises a belt retraction unit.

4. The medical securing unit of claim 3, wherein the belt stowing unit has a housing unit and the belt retraction unit is supported rotatably on the housing unit within a space enclosed by the housing unit.

5. The medical securing unit of claim 3, wherein the belt retraction unit has at least one retraction element configured to generate a torque.

6. The medical securing unit of claim 5, wherein the at least one retraction element comprises a spring element.

7. The medical securing unit of claim 3, wherein the belt retraction unit has at least one cylindrical rolling body.

8. The medical securing unit of claim 5, wherein the cylindrical rolling body has at least one internal hollow space and the at least one retraction element is disposed within the hollow space.

9. The medical securing unit of claim 5, wherein the at least one retraction element has two opposing end regions along its longitudinal extension and a first end region of the at least one retraction element is disposed on the cylindrical rolling body and a second end region is disposed on a housing unit of the belt stowing unit.

10. The medical securing unit of claim 5, wherein the belt retraction unit has at least two retraction elements that are disposed one behind the other within the cylindrical rolling body.

11. The medical securing unit of claim 10, wherein a first retraction element of the at least two retraction elements is configured differently from a second retraction element of the at least two retraction elements with respect to a physical property, a material property, or both a physical property and a material property.

12. The medical securing unit of claim 3, wherein the belt stowing unit has at least one locking element by which a retraction position of the belt retraction unit is locked.

13. The medical securing unit of claim 12, wherein the belt stowing unit has an actuating element, with a locking position of the at least one locking element being set as a function of an actuation position of the actuating element relative to a housing unit of the belt stowing unit.

14. The medical securing unit of claim 1, further comprising at least one fastening element.

15. The medical securing unit of claim 1, further comprising a housing unit with at least one marking element, the at least one marking element indicating an available belt length of the securing belt element.

16. A medical accessory unit comprising:
at least one medical securing unit to secure the position of a patient, a medical accessory unit, or both the patient and the medical accessory unit, the medical securing unit comprising at least one securing belt element and a belt stowing unit.

17. The medical accessory unit of claim 16, further comprising a fastening element configured to correspond to a fastening element of the at least one medical securing unit.

18. The medical accessory unit of claim 16, further comprising a housing unit that contains the at least one medical securing unit.

19. A patient support apparatus comprising:
at least one medical securing unit to secure the position of a patient, a medical accessory unit, or both the patient and the medical accessory unit, the medical securing unit comprising at least one securing belt element and a belt stowing unit.

20. The patient support apparatus of claim 19, further comprising a table configured to support a patient, the table having at least one fastening element configured to fasten the at least one medical securing unit.

21. The patient support apparatus of claim 20, wherein the fastening element has a fastening rail.

22. The patient support apparatus of claim 19, further comprising a table configured to support a patient, the table comprising the at least one medical securing unit.

23. A medical imaging apparatus comprising:
a patient support apparatus comprising a table configured to support a patient (701); and
at least one medical securing unit with at least one securing belt element configured to secure the patient, a medical accessory unit on the table, or both the patient and the medical accessory unit, the at least one medical securing unit comprising at least one securing belt element and a belt stowing unit.

24. The medical imaging apparatus of claim 23, wherein the table comprises the at least one medical securing unit.

25. The medical imaging apparatus of claim 23, wherein the medical accessory unit comprises a housing unit that contains the at least one medical securing unit.
